(19) United States
(12) Patent Application Publication
(10) Pub. No.: US 2025/0137047 A1
(43) Pub. Date: May 1, 2025

(54) Direct Synthesis of Oligonucleotides on Microtomed Tissue Slices

(71) Applicant: Miltenyi Biotec B.V. & Co. KG, Bergisch Gladbach (DE)

(72) Inventors: Robert Pinard, Lowell, MA (US); Michel Perbost, Bergisch Gladbach (DE); Matthias Bernhard Wahl, Bergisch Gladbach (DE)

(21) Appl. No.: 18/837,700

(22) PCT Filed: Feb. 13, 2023
(86) PCT No.: PCT/EP2023/053437

§ 371 Date: Aug. 12, 2024

(30) Foreign Application Priority Data
Feb. 14, 2022 (DE) 10 2022 103 440.2

(51) Int. Cl. C12Q 1/6874; C12Q 1/6806; C12Q 1/6823; C12Q 1/6841; C12Q 1/6876

(52) U.S. Cl. CPC C12Q 1/6874; C12Q 1/6806; C12Q 1/6823; C12Q 1/6841; C12Q 1/6876

(57) ABSTRACT

The invention is directed to a method to synthesize oligonucleotides on the surface of a biological sample comprising the steps a. Binding a plurality of primer molecules to spatial locations on the surface of the biological sample with a stochastic surface distribution thereby creating a oligonucleotides bound to the biological sample b. providing the biological sample with A, T, C or G nucleotides having a protecting unit at their 3′ positions c. incorporating one of the A, T, C or G nucleotides having a protecting unit at their 3′ positions at the 3′end of at least one oligonucleotides bound to the biological sample by addition of a terminal transferase thereby extending the oligonucleotides d. adding at least one photo-activated cleave agent capable of removing the protection unit from the incorporated protected nucleotide e. removing the protecting unit from the incorporated protected nucleotide by activating the photo-activated cleave agent with light provided to at least one spatial location of the biological sample f. Repeating steps b) to e) to incorporate further nucleotides to at least one oligonucleotide.

### BACKGROUND

The invention relates to direct synthesis of oligonucleotides on tissue slices to add a spatially known barcode.

Methods using spatially known barcodes oligonucleotides were already described: these methods associate a pre-synthesized oligonucleotide sequence to a specific location. Typical DNA synthesis is not compatible with biological material such as tissue slides as it uses phosphoramidite chemistry with anhydrous conditions and organic solvents.

As they are pre-synthesized in a DNA synthesizer, the oligonucleotides need to be somehow “positioned” on the right location: the methods lack flexibility, and accuracy positioning is challenging.

On the other hand, water based oligonucleotide synthesis is known. It is performed using an enzyme, terminal transferase, with 3′protected nucleotide triphosphate. Several companies, such as DNA script and molecular assemblies, have commercialized kits and processes in this field.

Accordingly, the synthesis of 3′protected nucleotides as well as the chemicals methods for deprotection is known.

Object of the present invention was to provide a method for direct synthesis of oligonucleotides, preferable on a surface or on tissue with optionally obtaining the spatial information of the oligonucleotide relative to the surface or tissue.

### SUMMARY

The invention is directed to utilize a terminal transferase to synthesize, optionally to an existing primer, oligonucleotides with the same or a different sequence over a surface, a protein, an antibody, or any biological sample, or an extension barcode sequence directly on it. Terminal transferase, being an enzyme, is used in aqueous and biologically compatible condition and requires protected building blocks. The necessary deprotection step, as it is similar to similar step during DNA sequencing, is also biologically compatible.

Object of the invention is therefore a method to synthesize oligonucleotides on the surface of a biological sample comprising the steps
a. Binding a plurality of primer molecules to spatial locations on the surface of the biological sample with a stochastic surface distribution thereby creating a oligonucleotides bound to the biological sample
b. providing the biological sample with A, T, C or G nucleotides having a protecting unit at their 3′ positions
c. incorporating one of the A, T, C or G nucleotides having a protecting unit at their 3′ positions at the 3′end of at least one oligonucleotides bound to the biological sample by addition of a terminal transferase thereby extending the oligonucleotides
d. adding at least one photo-activated cleave agent capable of removing the protection unit from the incorporated protected nucleotide
e. removing the protecting unit from the incorporated protected nucleotide by activating the photo-activated cleave agent with light provided to at least one spatial location of the biological sample
f. Repeating steps b) to e) to incorporate further nucleotides to at least one oligonucleotide.

The specific locations of a barcode can be created either by cleaving locally the 3′protecting group, for further elongation, using a photoactivable cleaving agent, such as a protected phosphine, or by physically separating the 4 nucleotides in various locations on the tissue.

### BRIEF DESCRIPTION OF THE DRAWINGS

FIG. 1-3 show the general method of the invention

### DETAILED DESCRIPTION

In the present invention, light is used to activate spatially the deprotection chemical for a spatially controlled deprotection of nucleotides and further, for spatially controlled extending the resulting oligonucleotide. As result, oligonucleotides with a defined sequence can be added to defined locations on the surface of the sample.

Photo deprotection is a known subject, as for example disclosed by Vaughan et al, JACS, 2013, 135(4) 1197-1200 and is used in a different technology to quench fluorescence. Any such photo deprotection technique can be used in the present invention. For example, as photo-activated cleave agent TCEP can be used which is deactivated by a reaction on cyanine dyes, or molecules reacting similarly with phosphine.

In the method of the invention, preferably the A, T, C or G nucleotides having a protecting unit at their 3′ positions are provided as mixture.

Depending on the further use of the oligonucleotide, in addition to the A, T, C or G nucleotides, nucleotides that bind all nucleotides to may be incorporated by further providing inosine nucleotides (I) having a protecting unit at their 3′ positions.

Furthermore, the oligonucleotides may be in part provided with a plurality of thymine nucleotides (T), thereby creating an oligonucleotide with a poly-T sequence capable of binding m-RNA originating from the sample.

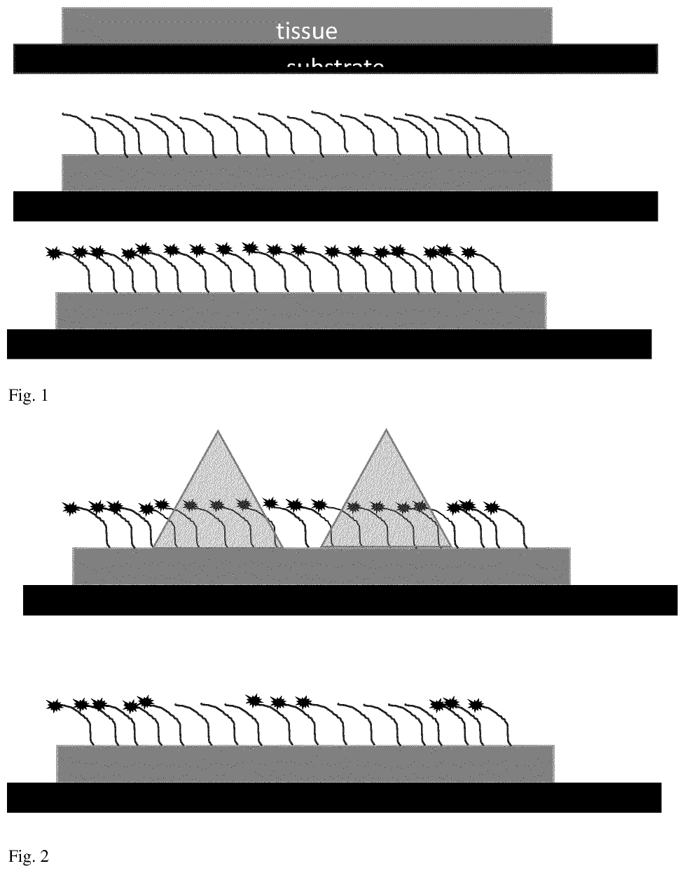

FIG. 1 shows the first steps of the method of the invention, where a tissue (biological sample) is placed on a surface and provided with primer molecules. The primer molecules are the starting unit of the oligonucleotides and may be provided with a protecting unit at their 3′ positions (shown as “*”). In an alternative variant, a terminal transferase is added and the primers are provided with A, T, C or G nucleotides having a protecting unit at their 3′ positions thereby extending the oligonucleotides. Again, the 3′ positions of the oligonucleotides are protected (shown as “*”).

The sample is then provided with at least one photo-activated cleave agent capable of removing the protection unit

Preferable, the A, T, C, G and optional I nucleotides having a protecting unit at their 3′ positions are provided subsequently and wherein after the step c), the unincorporated nucleotides are removed from the biological sample.

The nucleotides may be provided to the spatial location where the photo-activated cleave agent is activated with light. This is shown in FIG. 2, where the photo-activated cleave agent is activated with light provided to at least one spatial location of the biological sample (shown as grey triangle). This step removes the protecting unit at least one spatial location of the biological sample, leaving the oligonucleotides at these locations ready for extension with further nucleotides.

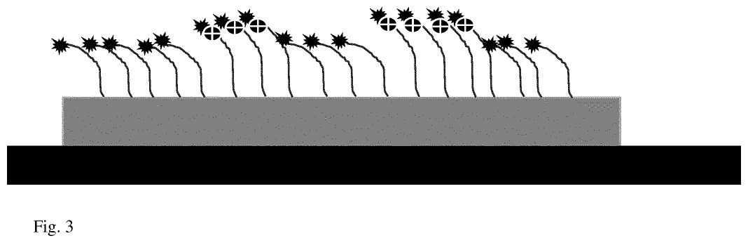

FIG. 3 shows that the thus unprotected oligonucleotides are extended by providing A, T, C or G nucleotides having a protecting unit at their 3′ positions in presence of a terminal transferase. On the thus deprotected sites new nucleotides (cross in circle), protected on 3′ (stars) are incorporated.

After each nucleotide addition (for instance A) and the optional following wash of its excess, the un-activated cleave solution is applied on to the entire surface.

Locations where the next nucleotides (for instance G) need to be coupled, will be exposed to a beam of light, photo-releasing the cleave agent. That cleave agent, in solution, will deprotect nearby 3′ protective groups. In these locations, the 3′end of the primers are deprotected, and can be elongated with the next nucleotide with terminal transferase.

Other locations, not exposed to light, would be still protected as the cleave agent was not deprotected. They will not be extended with a new nucleotide.

The number of oligonucleotides will be a function of the dimension of the laser beam, its accuracy and the method to control the diffusion of deprotected phosphine within that space. Because of the spatial controlled manner of the method, the oligonucleotides may serve as barcode information for further sequencing of the tissue, i.e. the barcode is “written” into the oligonucleotide in a spatial controlled manner.

This sequences of steps may be repeated as often as needed and at locations of the sample as desired, thereby extending the oligonucleotides in spatially controlled manner with a defined sequence.

In theory, the sequence may be repeated indefinitely. However, in praxis, steps b) to e) can be repeated 1 to 100 times to incorporate further nucleotides to at least one oligonucleotide.

In an embodiment of the invention, the nucleotides are added subsequently to the oligonucleotide at a first spatial location by successive adding then activating the photo-activated cleave agent with light provided to the first spatial location wherein the photo-activated cleave agent removes the 3′ protecting unit from the 3′end nucleotide and the deprotected 3′end nucleotide binds to a new nucleotide.

A common sequence to the entire tissue could be added to the localisation sequence. This sequence can be used as target sequence for a primer “B”. Primer “B” can have the sequence complementary to the sequence on the tissue extended by a sequence targeting a specific mRNA. The first primer can be further extended by a regular polymerase. When the duplex is formed, primer “B” can be eliminated (if dU was used to manufacture it, and with USER enzyme), and now primer A has the mRNA target sequence to capture mRNA.

After capture of the mRNA and its Reverse Transcription (RT), the newly formed cDNA will have the location sequence and the mRNA sequence, both can be sequenced separately.

In a variant of the method, the oligonucleotide is provided with a sequence of nucleotides coding for the spatial location of the oligonucleotide on the sample.

In another variant of the method, the oligonucleotide is provided with a sequence of nucleotides coding for the sample.

Preferable, after providing the surface of the biological sample with a plurality of primer molecules, the biological sample is imaged to obtain the spatial information of the location of the primer molecules.

The synthesis of oligonucleotides according to the method of the invention may be controlled by providing at least four photo-activated cleave agents which are activated by light having different wavelengths.

The oligonucleotides can be removed from the sample by providing photo-cleavable primer molecules

1. A method to synthesize oligonucleotides on the surface of a biological sample comprising the steps of:
a. binding a plurality of primer molecules to spatial locations on the surface of the biological sample with a stochastic surface distribution thereby creating a oligonucleotides bound to the biological sample;
b. providing the biological sample with A, T, C or G nucleotides having a protecting unit at their 3′ positions;
c. incorporating one of the A, T, C or G nucleotides having a protecting unit at their 3′ positions at the 3′end of at least one oligonucleotides bound to the biological sample by addition of a terminal transferase thereby extending the oligonucleotides;
d. adding at least one photo-activated cleave agent capable of removing the protection unit from the incorporated protected nucleotide;
e. removing the protecting unit from the incorporated protected nucleotide by activating the photo-activated cleave agent with light provided to at least one spatial location of the biological sample; and
f. repeating steps b) to e) to incorporate further nucleotides to at least one oligonucleotide.

2. The method according to claim 1 characterized in the A, T, C or G nucleotides having a protecting unit at their 3′ positions are provided as mixture.

3. The method according to claim 1 characterized in the A, T, C or G nucleotides having a protecting unit at their 3′ positions are provided subsequently and wherein after the step c), the unincorporated nucleotides are removed from the biological sample.

4. The method according to claim 1 characterized in that the nucleotides are provided to the spatial location where the photo-activated cleave agent is activated with light.

5. The method according to claim 1 characterized in that the oligonucleotide is provided with a sequence of nucleotides coding for the spatial location of the oligonucleotide on the sample.

6. The method according to claim 1 characterized in that the oligonucleotide is provided with a sequence of nucleotides coding for the sample.

7. The method according to claim 1 characterized in after providing the surface of the biological sample with a plurality of primer molecules, the biological sample is imaged to obtain the spatial information of the location of the primer molecules.

8. The method according to claim 1 characterized in further providing Inosine nucleotides having a protecting unit at their 3′ positions.

9. The method according to claim 1 characterized in providing at least four photo-activated cleave agents which are activated by light having different wavelengths.

10. The method according to claim 1 characterized in that the oligonucleotides are removed from the sample by providing photo-cleavable primer molecules.

11. The method according to claim 1 characterized in that the oligonucleotides are at least in part provided with a poly-T sequence capable of binding m-RNA originating from the sample.
